(19) United States
(12) Patent Application Publication
(10) Pub. No.: US 2005/0137717 A1
(43) Pub. Date: Jun. 23, 2005

(54) Prosthetic foot with rocker member

(76) Inventors: Finn Gramnas, Kinna (SE); Heidrun Gigja Ragnarsdottir, Reykjavik (IS); Sigurdur Asgeirsson, Gardabaer (IS)

Correspondence Address: KNOBBE MARTENS OLSON & BEAR LLP, 2040 MAIN STREET, FOURTEENTH FLOOR, IRVINE, CA, 92614, US

(21) Appl. No.: 10/742,455

(22) Filed: Dec. 18, 2003

(51) Int. Cl. A61F 2/62; A61F 2/66

(52) U.S. Cl. 623/38; 623/52; 623/55

(57) ABSTRACT

A prosthetic foot comprises a foot member, a rocker member connected in cantilever fashion to the foot member, and an ankle module movably connected to the rocker member about an axis, wherein an anterior section of the rocker member is configured to roll-up onto the foot member an amount corresponding to the load applied on the foot, and wherein the foot member is configured to facilitate the initial roll-up of the rocker member.

### BACKGROUND OF THE INVENTION

1. Field of the Invention

The present invention relates in one embodiment to lower limb prostheses in general, and, in particular, to a prosthetic foot having an ankle section with a rocker member connected to a foot member, where the rocker member facilitates the flexion of the foot member.

2. Description of the Related Art

Prosthetic feet of different designs are well known in the art. The various conventional designs have sought to solve various limitations associated with prosthetic feet.

Common to many conventional prosthetic foot designs is the desire to approximate the feel and fluid range of motion of a human foot's natural stride. One aspect of said natural stride is the ability to fluidly transition from heel-strike to toe-off during motion of the foot.

Some conventional designs attempt to provide said fluid transition by incorporating springs to store and release energy during motion of the prosthetic foot. The springs can be of different shapes, such as C-shaped or U-shaped, and of different types, such as leaf springs. However, such foot designs tend to be bulky and may be difficult to wholly contain in a cosmesis. Additionally, in some instances, the efficiency of the springs may deteriorate following prolonged use, resulting in less efficient energy storage and release during motion of the foot.

Other designs employ an ankle module pivotally connected to the foot member with bumpers disposed between each end of the ankle module and the foot member. In such designs, the bumpers store and release energy during heel-strike and toe-off. However, such designs also have disadvantages.

Additionally, existing prosthetic foot designs do not provide the desired degree of stride fluidity during foot motion. For example, existing designs do not adequately adapt the degree of flexion of the foot based on the load being applied to the foot. Such foot designs thus allow the same degree of flexion for lighter and heavier individuals, resulting in a less fluid foot motion for both individual types.

Some existing designs are difficult to fit into and remove from a cosmesis. Such ease of removal and introduction is particularly useful for performance of maintenance on the foot. For example, in foot designs that utilize bumpers, a user may want to replace the bumpers to vary the stiffness of the foot. Ease of removal of the foot from the cosmesis facilitates such replacement.

Accordingly, there is a need for an improved prosthetic foot that solves some of the problems discussed above.

### SUMMARY OF THE INVENTION

In at least one embodiment, the prosthetic foot is configured to provide an improved fluid transition between heel-strike and toe-off. The foot has a rocker member having an anterior section and a posterior section, wherein the anterior section is elongated in shape. The rocker member is preferably removably connected in cantilever fashion at the posterior section thereof to a foot member, wherein the foot member has a toe section and a heel section. In one embodiment, the posterior section of the rocker member has a generally planar lower surface, whereas the anterior section has a generally curved lower surface. In another embodiment, the front and posterior sections of the rocker member have generally planar lower surfaces.

As the foot transitions from heel-strike to toe-off, flexion of the foot member increases and the rocker member gradually rolls-up onto the foot member. This roll-up effect advantageously adjusts the stiffness of the foot to the load being applied. The greater the applied load, the more the rocker member rolls-up onto the foot member, and the greater the increase in the flexion and stiffness of the foot member. The stiffness of the foot member can also be varied based on the location along the foot member where the rocker member is connected. The further the rocker member is connected from the heel of the foot member, the greater the increase in stiffness during roll-up of the rocker member onto the foot member.

In one embodiment, the foot member is made of layers of composite material, wherein the layers define a certain foot member thickness. Optionally, the foot member may be tapered, for example, at the heel section. The taper and thickness of the lay-up design is preferably configured to provide a foot member designed for a target applied toe load. Additionally, the taper and thickness of the lay-up design are configured so that the rocker member rolls-up onto the foot member a desired amount when the target toe load is applied.

In another preferred embodiment, the anterior section of the rocker member is advantageously tapered for easier introduction into and removal from a cosmesis. Similarly, the posterior section of the rocker member can also be tapered. Moreover, the foot blade can also be tapered at the heel section thereof to facilitate the initial roll-up of the rocker member onto the foot member. Also, the length of the anterior section of the rocker member can be advantageously varied to provide an increased roll-up effect. In another embodiment, the anterior section can have a recessed or indented surface to decrease the weight of the rocker member.

In some embodiments, the prosthetic foot also comprises an ankle module having an anterior portion and a posterior portion, the module movably connected to the rocker member about an axis. Bumpers can be disposed between the ankle module and rocker member to provide energy storage and release during foot motion. Preferably, at least one of the bumpers is made of compressible material. Optionally, at least one of the bumpers can be made of a rigid or semi-rigid material. More preferably, at least a portion of the bumpers is compressible. In one embodiment, the bumpers are advantageously configured to reduce any clicking or other noise generated during the transition from heel-strike to toe-off of the foot. For example, at least one of the bumpers can be configured to function as a muffler or damper.

### BRIEF DESCRIPTION OF THE DRAWINGS

FIG. 1 is a perspective view of one embodiment of a prosthetic foot.

FIG. 2A is a side elevational view of the prosthetic foot shown in FIG. 1 with the heel in a neutral position.

FIG. 2B is a side elevational view of the prosthetic foot in FIG. 1 having a foot member heel section with a tapered thickness.

FIG. 2C is an exploded side view of the prosthetic foot shown in FIG. 1.

FIG. 3 is a front elevational view of the prosthetic foot illustrated in FIG. 1.

FIG. 4 is a top elevational view of the prosthetic foot illustrated in FIG. 1.

FIG. 5 is a longitudinal cross-sectional view of one embodiment of an ankle module and rocker member.

FIG. 6 is a transverse cross-sectional view of the ankle module and rocker member shown in FIG. 5.

FIG. 7A is a side elevational view of the prosthetic foot shown in FIG. 1 attached to a pylon and having one heel height.

FIG. 7B is a side elevational view of the prosthetic foot in FIG. 7A at a different heel height.

FIG. 8A is a side elevational view of another embodiment of a prosthetic foot with a different ankle module.

FIG. 8B is a cross-sectional view of the proximal end of the ankle module shown in FIG. 8A.

FIG. 9A is a side elevational view of the prosthetic foot shown in FIG. 8A attached to a pylon and having one heel height.

FIG. 9B is a side elevational view of the prosthetic foot shown in FIG. 8A at a different heel height.

FIG. 10 is a side elevational view of the prosthetic foot in FIG. 7A in a flexed state.

### DETAILED DESCRIPTION OF THE PREFERRED EMBODIMENTS

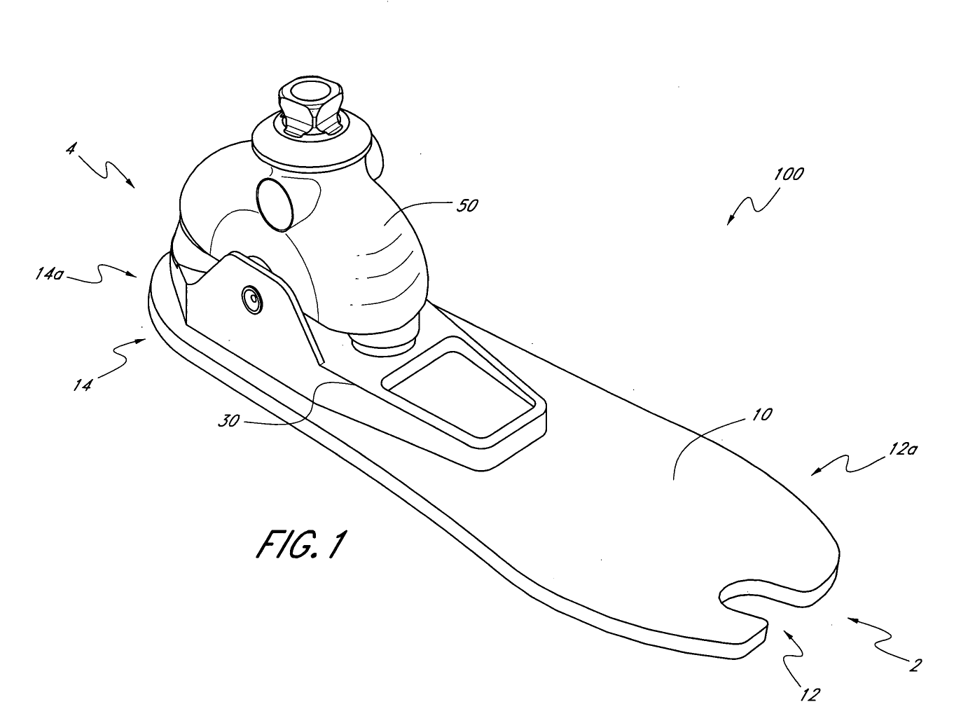

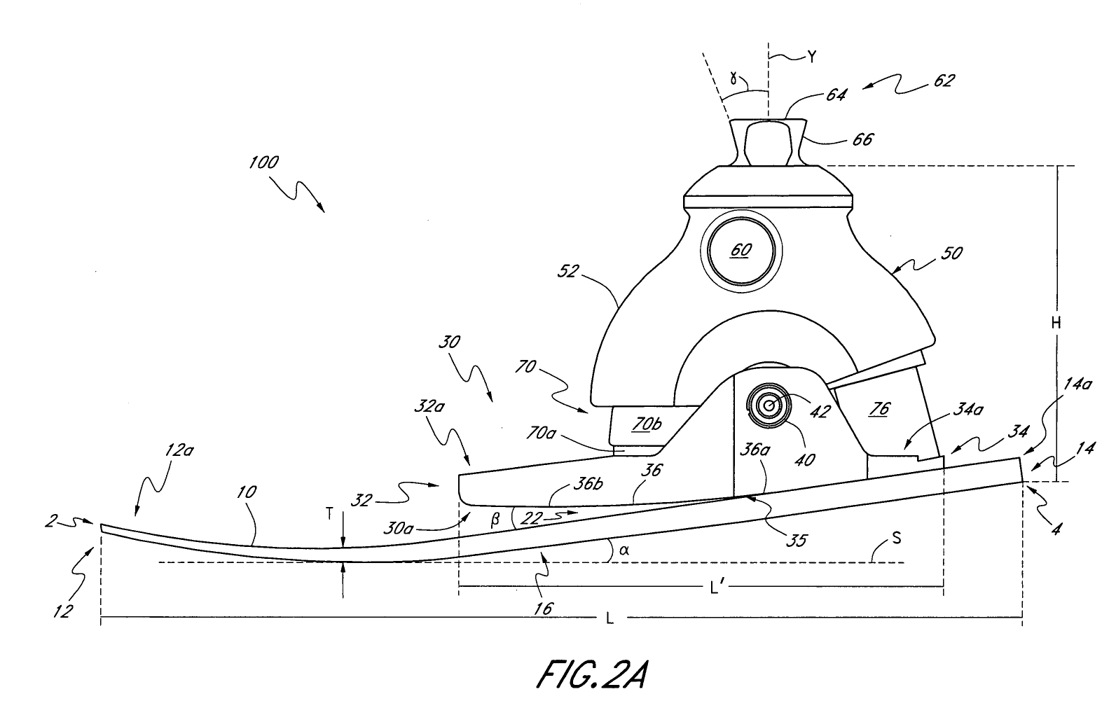

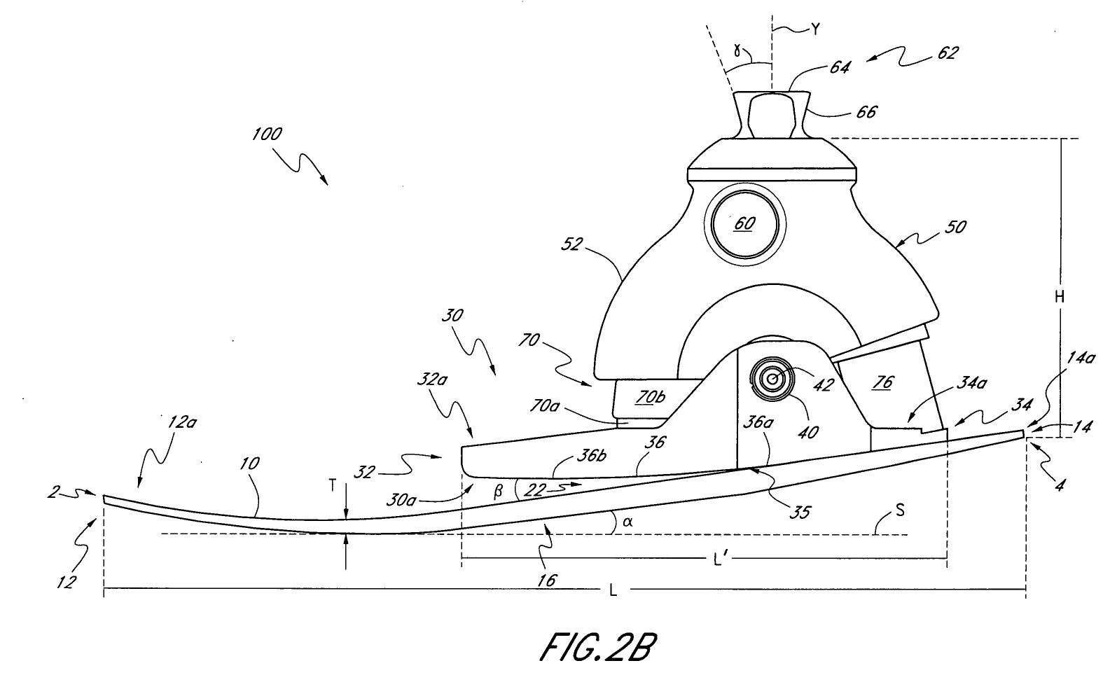

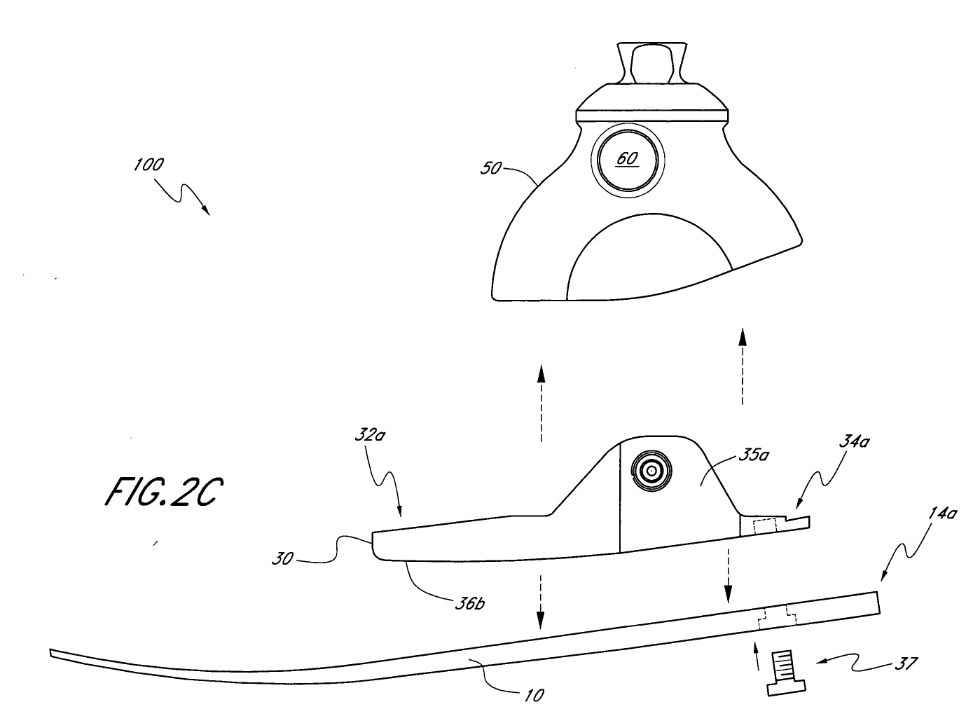

FIGS. 1-2A illustrate one embodiment for a prosthetic foot 100 extending between a toe section 2 and a heel section 4. Preferably, the prosthetic foot 100 comprises a foot member or support 10 which may have an elongate configuration having a length L extending between a front end 12 and a rear end 14. As used herein, length L refers to the horizontal length of the foot member 10 along a plane parallel to a support surface S on which the prosthetic foot 100 rests. Preferably, the length L can be between about 18 and 40 cm, corresponding to the size of the prosthetic foot 100, when the foot 100 has a neutral heel height position, as described below. In one embodiment, the length L is about 25 cm. However, the length L can have other values and can vary as the heel height position of the prosthetic foot 100 is adjusted. The foot member 10 also preferably comprises an anterior portion 12a, a posterior portion 14a, and an intermediate portion 16. In one embodiment, the anterior portion 12a can include a front toe portion configured to operatively contact the support surface S. The posterior portion 14a can comprise a heel portion, and the intermediate portion 16 can comprise an arch portion. Additionally, in one embodiment the foot member 10 can be generally shaped like the sole of the human foot, wherein the length L is approximately equal to that of a natural human foot. Alternatively, the foot member 10 may be shorter. In some embodiments, the foot member 10 may comprise multiple pieces separated, for example, transversely or longitudinally from each other. In other embodiments, the foot member 10 may be an integral piece, may be substantially flat, and may have a substantially rectangular traverse cross-section along its length L.

The foot member 10 as shown in FIG. 2A is preferably made of a material adapted to flex during motion from heel-strike through toe-off and has the desired strength. In one embodiment, the foot member 10 can be fabricated using a carbon filament with a polymer binder, for example, epoxy. However, other filament types can be used, such as glass, Kevlar, and nylon to ensure lightweight and structural and dynamic characteristics consistent with the amputee. Preferably, the foot member 10 is constructed using a combination of longitudinal (lengthwise) filaments interspersed with a fraction of transverse filament to bind the longitudinal filaments together and prevent the separation thereof under load. For example, in one embodiment, longitudinal or 90-degree filament, and transverse or 0-degree filament, can be used. However, in other embodiments, the longitudinal and transverse filaments can be arranged in other configurations, such as at 45 degrees relative to each other. Preferably, the longitudinal and transverse filaments are arranged together with the polymer binder in laminae that are located in immediate contact with one another. For example, the laminae can be superimposed on each other, maintained in operative relationship by the polymer binder, or additionally by encapsulating polymer or filaments arranged in the thickness direction, and be susceptible to a bending stress determined by the thickness of the superimposed laminae. The number of laminae preferably varies with the size of the prosthetic foot 100. For example, the foot member 10 of a smaller prosthetic foot 100 can comprise a lower number of laminae than the foot member 10 of a larger prosthetic foot 100. Accordingly, a thickness T of the foot member 10 will vary with the number of laminae used to fabricate the foot 100. Further details of material suitable for use in fabricating the foot member 10 can be found in U.S. Pat. Nos. 4,547,913 and 4,822,363, both of which are hereby incorporated by reference.

The foot member 10 in FIG. 2A can be fabricated using, for example, injection molding and/or the use of thermoplastic materials and processes or any of a range of combinations thereof. In one preferred embodiment, chopped fiber may be blended in a thermoplastic or a thermoset resin and the resulting mixture injection molded into an appropriate configuration. In another preferred embodiment, thermoplastic or thermoset laminae may be alternatively or additionally wound around an injection molded core or a thermoplastic resin may be injected between thermoplastic or thermoset laminae whereby the laminates are bonded onto the injected material.

The foot member 10 in one embodiment is a generally flat plate-like member, which may or may not have some curvature. As shown in FIG. 2A, the posterior and intermediate sections 14a, 16 are generally planar while the anterior section 12a is generally curved. The posterior and intermediate sections 14a, 16 are generally inclined with respect to the support surface S. For example, the posterior portion 14a can extend at an angle α relative to the support surface S. The angle α can be between about 10 and 30 degrees when the foot 100 is at rest and has a neutral heel height position, as described below. In the illustrated embodiment, the angle α is about 15 degrees with the foot 100 at rest and in a neutral heel height position. However, the angle α can have other values and can vary during motion of the prosthetic foot 100 and when the heel height position of the foot 100 is adjusted. In another embodiment, the foot member 10 can be generally planar and extend substantially parallel to the support surface S from the toe section 2 to the heel section 4. In another embodiment, the posterior and intermediate sections may also be curved.

As shown in FIG. 2A, the thickness T of the foot member 10 tapers between a maximum at the rear end 14 and a minimum at the front end 12. In some embodiments, the thickness T can vary from between about 7 and 10 millimeters at the rear end 14 to between about 2.5 and 5 millimeters at the front end 12. In one embodiment, the thickness T varies between about 8 millimeters at the rear end 14 to about 3 millimeters at the front end 12. In another embodiment (not shown), the thickness T of the foot member 10 may be uniform from the rear end 14 to the front end 12. In one embodiment, the foot member 10 has a uniform thickness T of about 7 mm. In still another embodiment, the thickness T of the foot member 10 can taper between a maximum at the intermediate section 16 and minimums at the front and the rear ends 12, 14, as shown in FIG. 2B.

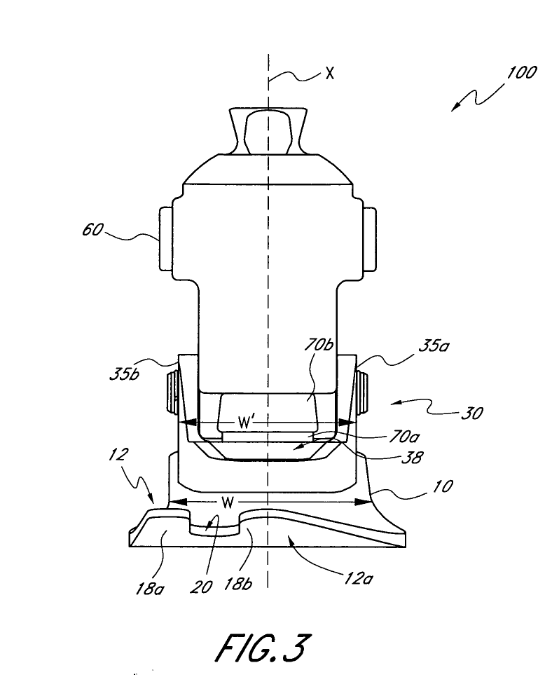

As best seen in FIG. 3, in one preferred embodiment, the anterior portion 12a of the foot member 10 comprises at least two toe members 18a, 18b. The toe members 18a, 18b are preferably defined by at least one longitudinal slot 20 in the foot member 10 extending rearwardly from the front end 12. In the illustrated embodiment, the longitudinal slot 20 extends into the foot member 10 about two to three centimeters from the front end 12. However, in other embodiments, the slot 20 can extend further or less into the foot member 10. In the illustrated embodiment, the longitudinal slot 20 is offset from a major axis X extending generally longitudinally along the midline of the foot member 10 to resemble either a left foot or a right foot. The left foot embodiment is illustrated in FIG. 3. In other embodiments, the longitudinal slot 20 can be substantially aligned with the axis X. In one embodiment, the slot 20 is adapted to receive a thong of a sandal or similar footwear. In another embodiment, the slot 20 is adapted to receive a foot cover (not shown) having a corresponding slot between the toe members 18a, 18b to provide a more aesthetically pleasing foot cover, or a cover adapted to receive a thong of a sandal or similar footwear.

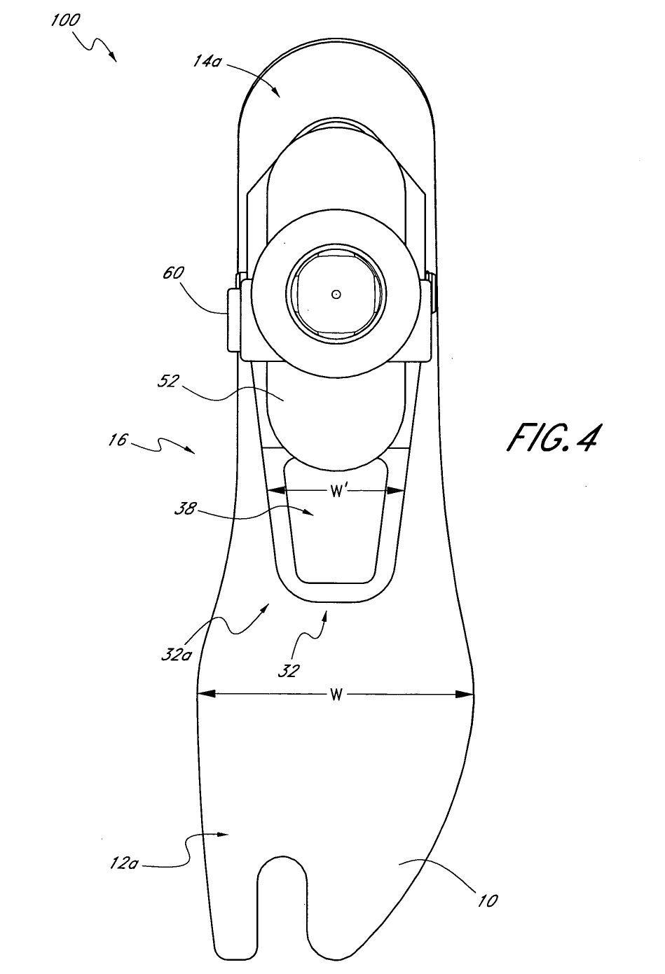

FIG. 4 shows a top view of the prosthetic foot 100 illustrated in FIG. 2A. In the illustrated embodiment, the posterior section 14a of the foot member 10 is tapered relative to the intermediate section 16 of the foot member 10 so that the posterior section 14a is less wide. For example, the foot member can have a width W that tapers toward the rear end 14. The anterior section 12a of the foot member 10 can also be tapered relative to the intermediate section 16. In one embodiment, the width W may taper gradually and continuously from the intermediate section 16 to the front and rear ends 12, 14. In another embodiment, the foot member 10 can have a generally constant width W along the intermediate section 16 and taper thereafter towards the front and rear ends 12, 14. In still another embodiment, the foot member 10 can have a generally constant width W from the front end 12 to the rear end 14.

As best shown in FIG. 2A, the prosthetic foot 100 also comprises a rocker member 30 mounted to the foot member 10 near the posterior section 14a of the foot member 10. The rocker member 30 can have an elongate configuration having a length L′ extending between a front end 32 and a rear end 34. In one embodiment, the length L′ is less than about 45% of the length L of the foot member 10. In another embodiment, the length L′ is about 45% or more of the length L of the foot member 10. In still another embodiment, the length L′ is about 50% or more of the length L of the foot member 10. In the illustrated embodiment, the length L′ is about 55% of the length L of the foot member 10. In a preferred embodiment, the front end 32 of the rocker member 30 extends past the transverse midline of the foot member 10. The length L′ is preferably between about 40 and 60 mm. In the illustrated embodiment, the length L′ is about 50 mm. The rocker member 30 also defines an anterior section 32a, a posterior section 34a, and an intermediate section 35. A base 36 extends along a lower portion 30a of the rocker member 30, from the posterior section 34a to the anterior section 32a.

The base 36 of the rocker member 30, as shown in FIG. 2A, defines a contact surface 36a that contacts the foot member 10 and a roll-up surface 36b that does not contact the foot member 10 when the prosthetic foot 100 is at rest. In the illustrated embodiment, the contact surface 36a extends generally along the posterior and intermediate sections 34a, 35 of the rocker member 30. In other embodiments, the contact surface 36a can extend along the posterior section 34a and partially along the intermediate section 35. In one embodiment, the contact surface 36a can extend solely along the posterior section 34a. In the illustrated embodiment, the roll-up surface 36b extends along the anterior section 32a, between the intermediate section 35 and the front end 32 of the rocker member 30. In other embodiments, the roll-up surface 36b can additionally extend partially along the intermediate section 35. In one embodiment, the roll-up surface 36b can extend generally along the anterior and intermediate sections 32a, 35.

As shown in the embodiment illustrated in FIG. 2A, the roll-up surface 36b is preferably curved. However, in other embodiments, the roll-up surface 36b can be generally planar. In one embodiment, the roll-up surface 36b can have a radius of curvature (not shown) corresponding to a radius of curvature (not shown) of the intermediate section 16 of the foot member 10. As illustrated, the anterior tip of the roll-up surface 36b may be rounded.

The contact surface 36a is preferably configured to mate with the surface of the posterior section 14a of the foot member 10. In one embodiment the contact surface 36a can also be curved and have a radius of curvature. In one embodiment, the radius of curvature of the contact surface 36a can be equal to the radius of curvature of the roll-up surface 36b. In another embodiment, the contact surface can be generally planar.

As shown in the embodiment illustrated in FIG. 2A when the rocker member 30 is connected to the foot member 10 and the foot 100 is at rest, the roll-up surface 36b extends relative to the contact surface 36a and the foot member 10 so as to define a longitudinal slot 22 between the anterior section 32a of the rocker member 30 and the foot member 10. For example, in one embodiment, the roll-up surface 36b can extend generally at an angle β relative to the contact surface 36a and to the posterior section 14a of the foot member 10. In another embodiment (not shown), where the roll-up surface 36b is generally planar, the roll-up surface 36b can be inclined relative to the foot member 10, but not relative to the contact surface 36a. The angle β is preferably between about 10 and 20 degrees. In the illustrated embodiment, the angle β is about 15 degrees.

In one embodiment, as shown in FIG. 2C, the rocker member 30 can be removably mounted to the foot member 10 via at least one connector 37. In some embodiments, the connector 37 may comprise one or a plurality of bolts connecting the foot member 10 to the posterior section 34a of the rocker member 30. However, the connector 37 can comprise other structures, such as rivets and screws. In other embodiments, the rocker member 30 can be permanently or releasably fixed to the foot member 10 via, for example, adhesives, straps, resins or welds. In one embodiment, the rocker member 30 is connected to the foot member 10 in cantilever form. Accordingly, the anterior section 32a of the rocker member 30 can move relative to the foot member 10, and the roll-up surface 36b can roll-up onto the foot member 10, during motion of the prosthetic foot 100. In one embodiment, such a cantilever connection can be achieved by connecting the rocker member 30 to the foot member 10 solely at the posterior section 34a of the rocker member 30.

The prosthetic foot 100 shown in FIG. 2A can have a roll-up surface 36b that is about 10% or more of the length L′ of the rocker member 30. In another embodiment, the length of the roll-up surface 36b can be about 20% or more of the length L′ of the rocker member 30. In still another embodiment, the length of the roll-up surface 36b can be about 30% or more of the length L′ of the rocker member 30. In yet another embodiment, the length of the roll-up surface 36b can be about 40% or more of the length L′ of the rocker member 30. In another embodiment, the length of the roll-up surface 36b can be about 50% or more of the length L′ of the rocker member 30. In another embodiment, the length of the roll-up surface 36b can be about 70% or more of the length L′ of the rocker member 30. In the illustrated embodiment, the length of the roll-up surface 36b is about 60% of the length L′ of the rocker member 30.

As best shown in FIGS. 3-4, the rocker member 30 has a width W′ that varies between a maximum at the intermediate section 35 to minimums at the anterior and posterior sections 32a, 34a. For example, in one embodiment, the width W′ may taper at a constant angle from the intermediate section 35 to the anterior and posterior sections 32a, 34a. In another embodiment, the width W′ may taper at a gradually increasing slope between the intermediate section 35 and the anterior and posterior sections 32a, 34a, such as in the form of a curve. In one embodiment, the width W′ tapers from about 4 cm at the intermediate section 35 to about 2 cm at the anterior section 32a and about 1 cm at the posterior section 34a. In yet another embodiment, the width W′ of the rocker member 30 may be generally uniform between the front and rear ends 32, 34 of the rocker member 30.

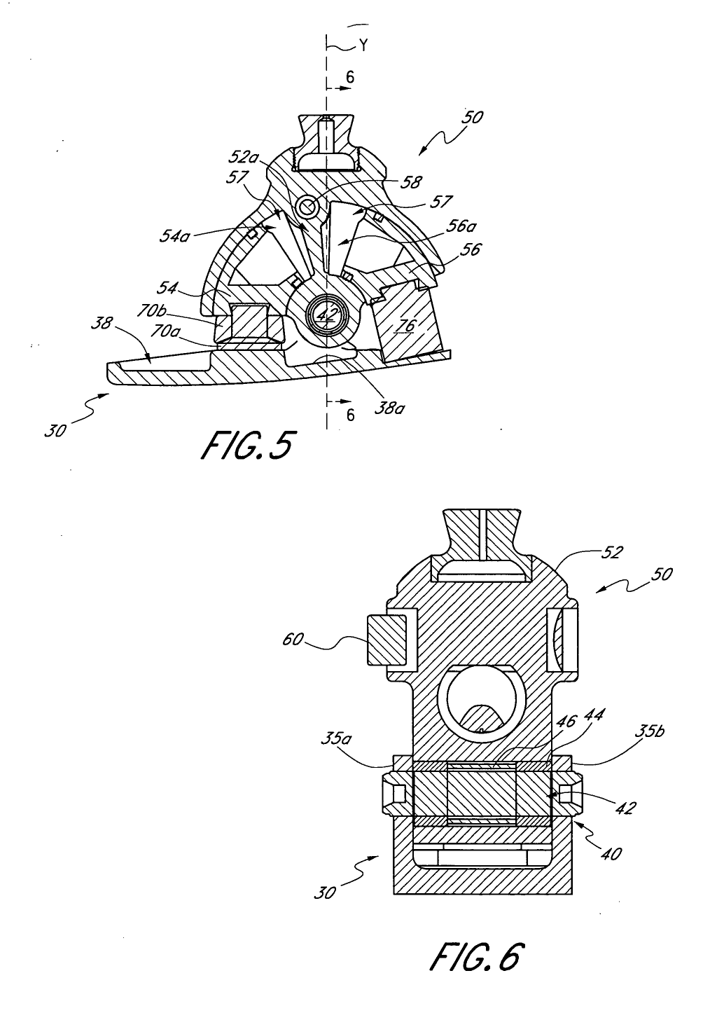

As best seen in FIGS. 4-5, the rocker member 30 can have recessed sections formed thereon configured to lower the weight of the rocker member 30. For example, in one embodiment, the anterior section 32a of the rocker member 30 may have a recessed portion 38 formed on an upper surface thereof. In another embodiment, the intermediate section 35 may have a recessed section 38a formed on an upper surface thereof.

As seen in FIG. 3 and FIG. 6 the intermediate section 35 of the rocker member 30 defines two opposite walls 35a, 35b which preferably extend generally parallel to each other. The rocker member 30 also defines an axial opening 40 formed on both walls 35a, 35b and adapted to receive an axle 42 therethrough. The axle 42 is configured to connect an ankle module 50 to the rocker member 30 such that the ankle module 50 is capable of pivoting about the axle 42 between the anterior and posterior sections 32a, 34a of the rocker member 30. At least one bearing 44 can be disposed between the axle 42 and the ankle module 50. In the embodiment illustrated in FIG. 6, two bearings 44 are disposed between the axle 42 and ankle module 50, with a bearing spacer 46 disposed between the two bearings 44.

As shown in FIGS. 2A and 5, the ankle module 50 in one embodiment comprises a housing 52 defining an anterior cylinder 54a and a posterior cylinder 56a therein. In one embodiment, the cylinders 54a, 56a have the same length. As shown in FIG. 2A, the ankle module 50 is operatively connected to the foot member 10 via rocker member 30. The anterior and posterior cylinders 54a, 56a are preferably sized to slidingly receive an anterior piston 54 and a posterior piston 56, respectively. In one embodiment, the pistons 54, 56 have the same length. Additionally, each of the cylinders 54a, 56a is preferably longer than its corresponding piston 54, 56 and defines a gap 57 between the piston 54, 56 and a wall 52a of the housing 52. Preferably, the anterior piston 54 is aligned with the anterior section 32a of the rocker member 30. Likewise the posterior piston 56 is preferably aligned with the posterior section 34a of the rocker member 30. The housing 52 also comprises a valve 58 disposed on the wall 52a and between the cylinders 54a, 56a. The valve 58 can be operated to selectively permit communication between the cylinders 54a, 56a. In the illustrated embodiment, the valve 58 is a spool valve. However, other valve types can be used.

When the valve 58 shown in FIG. 5 is in an open position, such that the cylinders 54a, 56a communicate with each other, a fluid contained in the cylinders 54a, 56a can flow between the cylinders 54a, 56a. The housing 52 can thus move relative to the pistons 54, 56 and be selectively pivoted about the axle 42 to a different position relative to a generally vertical axis Y. When the valve 58 is in a closed position, there is no communication between the cylinders 54a, 56a so that the fluid cannot flow between the cylinders 54a, 56a. As a result, the housing 52 cannot move relative to the pistons 54, 56 and is held in a substantially fixed position relative to the vertical axis Y.

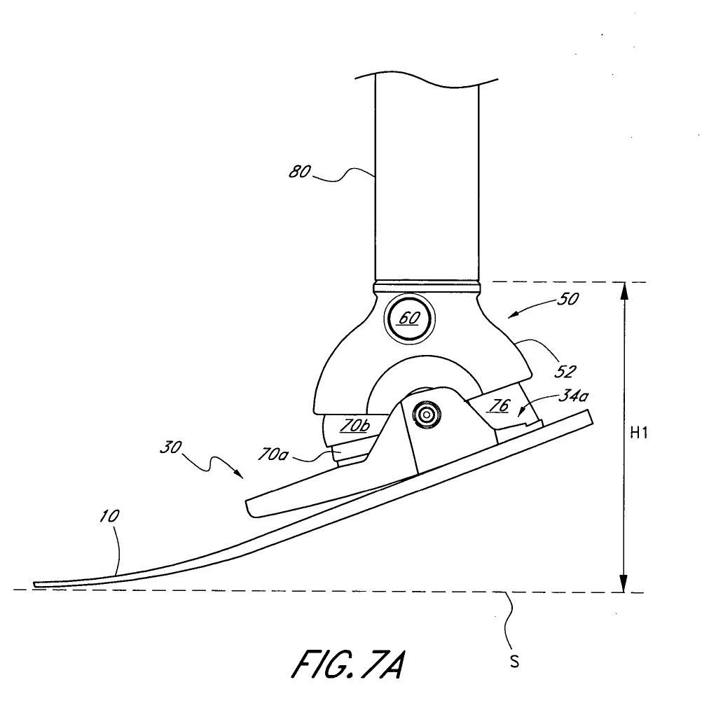

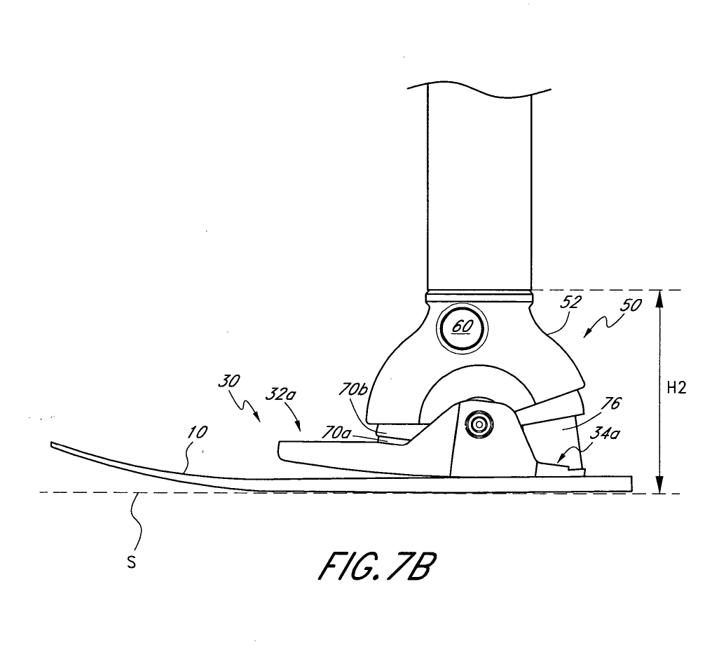

In one embodiment, the volume of the gap 57 in each cylinder 54a, 56a, as shown in FIG. 5, will preferably vary when the valve 58 is in the open position and the housing 52 is moved relative to the pistons 54, 56 to a different position. For example, when the heel of the foot 100 is in a neutral heel height position, the gap 57 in each cylinder 54a, 56a generally has the same volume. However, when the housing 52 is rotated toward the anterior section 32a of the rocker member 30 (see e.g., FIG. 7B) to achieve a lower heel height, the gap 57 in the anterior cylinder 54a will have a lower volume than the gap 57 in the posterior cylinder 56a. Likewise, when the housing 52 is rotated toward the posterior section 34a of the rocker member (see e.g., FIG. 7A) to achieve a higher heel height, the gap 57 in the anterior cylinder 54a will have a greater volume than the gap 57 in the posterior cylinder 56a.

The valve 58 shown in FIG. 5 may be actuated in one of a variety of ways. In the embodiment illustrated in FIG. 2A, a push button 60 is adapted to move upon receipt of a force to actuate the valve 58 between an open and a closed position. Said actuation allows the movement and fixation of the housing 52 relative to the pistons 54, 56 to reposition the ankle module 50 relative to the axis Y, as described above. However, other actuation mechanisms may be used, such as a lever (not shown). Further details of the ankle module 50 can be found in U.S. Pat. No. 5,957,981, which is hereby incorporated by reference in its entirety.

As best shown in FIG. 2A a front bumper 70 is disposed between the anterior piston 54 and the anterior section 32a of the rocker member 30. Similarly a rear bumper 76 is disposed between the posterior piston 56 and the posterior section 34a of the rocker member 30. In some embodiments, one or both of the bumpers 70, 76 can be removably disposed between the ankle module 50 and the rocker member 30.

In the embodiment illustrated in FIG. 2A, the front bumper 70 comprises a generally compressible portion 70a and a generally rigid portion 70b. In one embodiment, the generally compressible portion 70a can be about 2 cm long. The compressible portion 70a and rigid portion 70b can be made of materials having different durometers. For example, in one embodiment, the compressible portion 70a can have a durometer of 95 Shore A and the rigid portion 70b can have a durometer of 70 Shore D. In another embodiment, the entire length of the front bumper 70 is incompressible.

Similarly, the rear bumper 76 shown in FIG. 2A can be made of a generally compressible material, such as a hard rubber or polyurethane, having one of a variety of durometers. In one embodiment, a kit can be provided comprising a plurality of rear bumpers 76, wherein each rear bumper 76 has a different durometer varying between a soft and an extra firm consistency. For example, the durometer of the rear bumper 76 can vary between 60 Shore A and 95 Shore A.

Preferably, the bumpers 70, 76 shown in FIG. 2A can be replaced. In one embodiment, the bumpers 70, 76 can be removed by applying a force to rotate the housing 52 of the ankle module 50 to expose the space between the pistons 54, 56 and the bumpers 70, 76. The bumpers 70, 76 can then be replaced with new bumpers or bumpers having a different durometer. For example, to remove the front bumper 70, the actuator 60 can be actuated to allow rotation of the housing 52 toward the posterior section 34a of the rocker member 30 via application of a force, thus exposing a space between the anterior piston 54 and the front bumper 70. The front bumper 70 can then be removed from between the anterior piston 54 and the anterior section 32a of the rocker member 30. Following replacement of the front bumper 70, a user can apply a force to rotate the housing 52 toward the anterior section 32a of the rocker member 30. Similarly, the actuator 60 can be actuated to allow rotation of the housing 52 toward the anterior section 32a of the rocker member 30 via application of a force, to expose a space between the posterior piston 56 and the posterior section 34a of the rocker member 30. The rear bumper 76 can then be removed from between the posterior piston 56 and the posterior section 34a of the rocker member 30. Following replacement of the rear bumper 76, the user can apply a force to the housing 52 to rotate the housing 52 toward the rear section 34a of the rocker member 30.

As illustrated in FIG. 2A, the ankle module 50 comprises a pyramid 62 having a top surface 64 and a side surface 66 and configured to receive a pylon or other prosthesis thereon. In the illustrated embodiment, the side surface 66 consists of a plurality of generally flat faces extending an angle γ relative to the generally vertical axis Y. In another embodiment the side surface 66 can comprise a generally cylindrical surface also extending at said angle γ. In other embodiments the side surface 66 can extend generally parallel to the vertical axis Y. As described above, the ankle module 50 can be selectively moved relative to the axis Y by actuating the valve 58 to allow the housing 52 to rotate relative to the pistons 54, 56. However, the pyramid 62 is to be oriented generally vertically when connected to a user's pylon or prosthesis. Accordingly, the movement of the housing 52 relative to the axis Y effectively adjusts the heel height of the prosthetic foot 100, wherein the heel height is defined as the distance between the support surface S and the pyramid 62. If the ankle module 50 is moved toward the posterior section 34a of the rocker member 30, the heel height is increased. Similarly, if the ankle module 50 is moved toward the anterior section 32a of the rocker member 30, the heel height is decreased.

FIG. 7A shows the prosthetic foot 100 with a pylon 80 attached to the ankle module 50. The pylon 80 can be cylindrical in shape, or have any other suitable shape, and be made of any material suitable for use in prosthetic pylons. In the illustrated embodiment, the ankle module 50 has been moved toward the posterior section 34a of the rocker member 30 so that the housing 52 rotates relative to the pistons 54, 56. As a result, the heel height from the support surface S to the junction of the pylon 80 and the ankle module 50 is increased to a heel height H1. In this configuration, a user can advantageously use the prosthetic foot 100 with shoes that have high heels.

FIG. 7B shows another configuration of the prosthetic foot in FIG. 7A with a different heel height. In the illustrated embodiment, the ankle module 50 has been moved toward the anterior section 32a of the rocker member 30 so that the housing 52 rotates relative to the pistons 54, 56. As a result, the heel height from the support surface S to the junction of the pylon 80 and the ankle module 50 is decreased to a heel height H2. In this configuration, a user can advantageously use the prosthetic foot with shoes that have low heels or when walking barefoot.

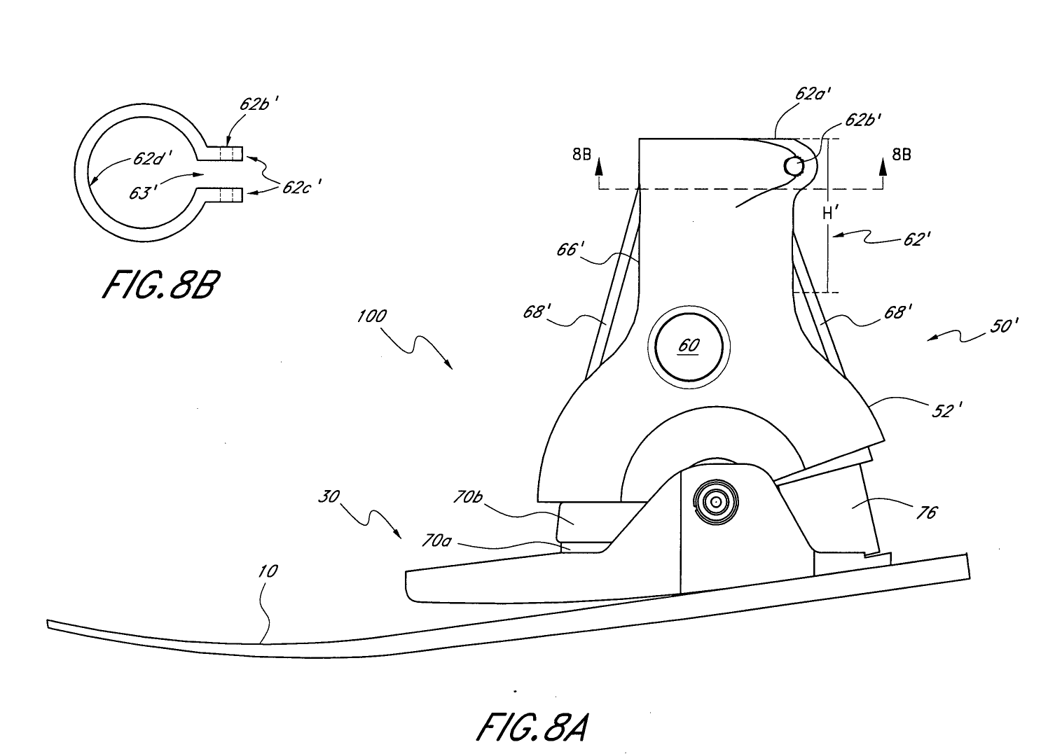

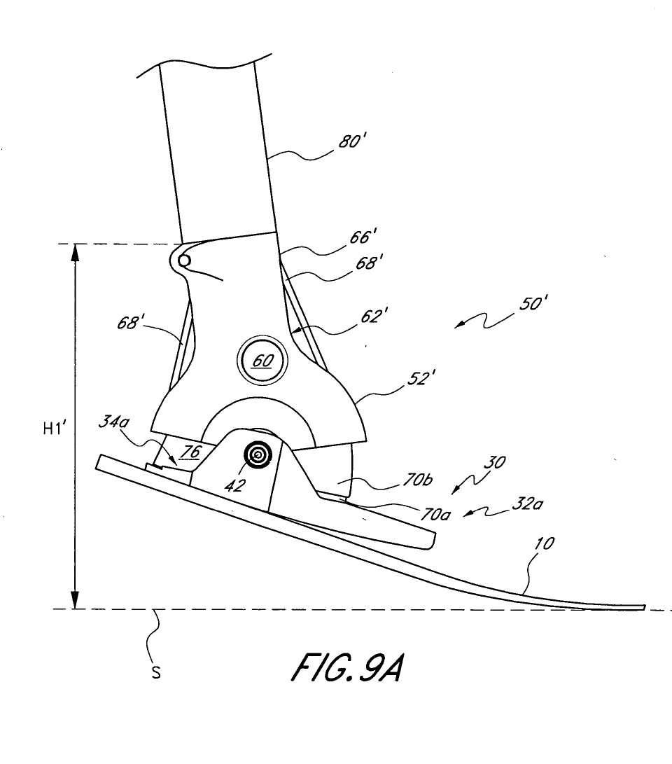

FIG. 8A shows another embodiment of the prosthetic foot 100 wherein the ankle module 50′ comprises a tube clamp 62′ having a generally cylindrical body 66′. The tube clamp 62′ preferably extends a height H′ from the housing 52′ to an upper end 62a′ and may be integrally formed with the housing 52′. Optionally, the tube clamp 62′ can have at least one support member extending between the upper end 62a′ and the housing 52′. In the illustrated embodiment, the tube clamp 62′ has two support members 68′. The tube clamp 62′ is configured to receive a pylon or other prosthesis therein. The clamp 62′ comprises a clamp bore 62b′ that extends through two clamp arms 62c′, as shown in FIG. 8B. The clamp arms 62c′ define a slot 63′ therebetween. The clamp bore 62b′ is configured to receive a connector (not shown) therethrough, such as a bolt or the like, to urge the clamp arms 62c′ toward each other. Accordingly, by urging the clamp arms 62b′ toward each other, an inner surface 62d′ of the clamp tube 62′ can be clamped about a surface of a pylon or other prosthesis.

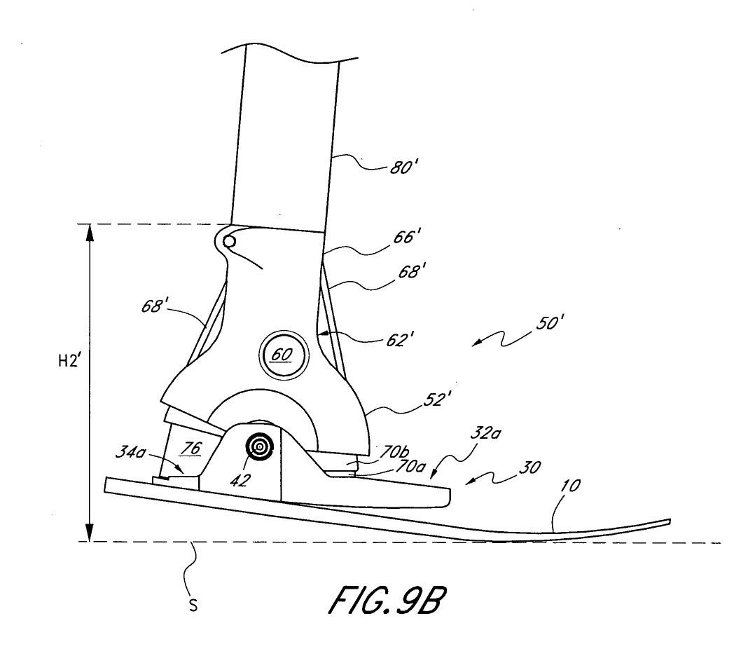

The embodiment of the prosthetic foot 100 shown in FIG. 8A can also be adjusted to provide different heel heights. FIG. 9A shows one embodiment of the prosthetic foot 100 with a pylon 80′ attached to the tube clamp 62′ of the ankle module 50′. In the illustrated embodiment, the ankle module 50′ has been moved toward the posterior section 34a of the rocker member 30 so that the housing 52′ rotates relative to the pistons 54, 56, as described above. As a result, the heel height from the support surface S to the junction of the pylon 80′ and the ankle module 50′ is increased to a heel height H1′. In this configuration, a user can advantageously use the prosthetic foot 100 with shoes that have high heels.

FIG. 9B shows another configuration of the prosthetic foot in FIG. 9A with a different heel height. In the illustrated embodiment, the ankle module 50′ has been moved toward the anterior section 32a of the rocker member 30 so that the housing 52′ rotates relative to the pistons 54, 56 (as shown in FIG. 5). As a result, the heel height from the support surface S to the junction of the pylon 80′ and the ankle module 50′ is decreased to a heel height H2′. In this configuration, a user can advantageously use the prosthetic foot with shoes that have low heels.

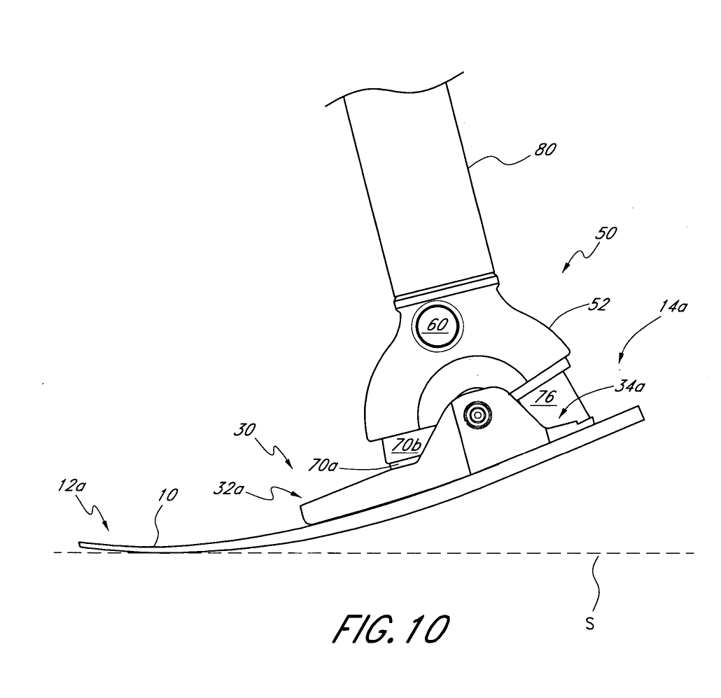

The prosthetic foot 100 of FIGS. 1-9B advantageously provides a fluid heel-to-toe movement as compared to other prosthetic foot designs. As a user proceeds from heel-strike to toe-off, the rocker member 30 rolls up onto the foot member 10, causing the posterior portion 14a of the foot member 10 to flex toward the anterior section 12a, as illustrated in FIG. 10. In the embodiments described in FIGS. 1-10 above, the roll-up is provided because the anterior section 32a of the rocker member 30 is free to move relative to the foot member 10, so that the roll-up surface 36b rolls-up onto the foot member 10. The roll-up effect provides flexion and gradual stiffening of the foot member 10 in comparison to conventional prosthetic foot designs, thus providing an improved fluid foot motion. The roll-up effect also facilitates more efficient energy storage and release during heel-strike through toe-off. The initial roll-up of the rocker member 30 onto the foot member 10 is further facilitated in embodiments where the thickness T of the foot member 10 is tapered at the posterior section 14a (see FIG. 2B) because the tapered thickness provides a lower resistance to flexion.

Advantageously, the degree of roll-up of the foot 100 shown in FIGS. 1-10 can be varied by varying the length of the roll-up surface 36b. As noted above, in the illustrated embodiment, the roll-up surface 36b is about 60% of the length L′ of the rocker member 30. However, the roll-up surface 36b can be about 10% or more of the length L′. Additionally, in the illustrated embodiment, the length L′ of the rocker member 30 is advantageously greater than about 50% of the length L of the foot member 10, which also contributes to an increased roll-up effect. Accordingly, in at least one embodiment, the front end 32 of the rocker member 30 extends forward of a midline of the foot member 10.

As discussed above with respect to FIG. 4, the foot member 10 can have a width W that tapers toward the posterior section 14a, or heel section 4, of the foot member 10. Such a tapered posterior section 14a or heel section 4 advantageously facilitates the roll-up of the rocker member 30 onto the foot member 10 during motion of the foot 100.

As best shown in FIGS. 2A, 2B, 5 and 7A-10, the bumpers 70, 76 are advantageously disposed between the rocker member 30 and the housing 52. In embodiments where the rocker plate 30 is removably mounted on the foot member 10, such a configuration also allows the ankle module 50, bumpers 70, 76, and rocker member 30 to be detached from the foot member 10 as a unit. Therefore this configuration advantageously facilitates the assembly and disassembly of the prosthetic foot 100, as well as the replacement of the bumpers 70, 76.

As noted above, the front bumper 70 shown in FIG. 2A advantageously comprises a generally compressible portion 70a and a generally rigid portion 70b. The front bumper 70 is thus adapted to operate as a muffler or damper, substantially preventing a “clicking” noise when the rear bumper 76 is compressed and released during motion of the foot 100.

Further, the rocker member 30, as shown in FIG. 3, can advantageously be tapered in width toward the front end 32 thereof, facilitating insertion and removal of the rocker member 30 from a cosmesis or foot cover. Additionally, the anterior section 32a of the rocker member 30 can have a recessed section 38 formed thereon, advantageously reducing the weight of the rocker member 30.

As noted above, the prosthetic foot 100 of FIGS. 1-10, can advantageously be adjusted to different heel heights. This allows the prosthetic foot 100 to be used in conjunction with a variety of footwear, each having a different heel height. Additionally, the adjustable heel height of the prosthetic foot 100 provides a user with increased comfort and security when walking up or down hills by allowing the user to adjust the orientation of the foot 100 as best suited for the grade of the hill's incline.

In FIGS. 1-10, as the user proceeds from heel-strike to toe-off, the pylon 80 or other prosthesis applies a forward force onto the housing 52 of the ankle module 50. However, when the valve 58 is in a closed position, there is no communication between the cylinders 54a, 56a. Accordingly, the housing 52 is not allowed to move relative to the pistons 54, 56. Instead, the wall 52a of the housing 52 hydraulically transfers said force to the anterior piston 54, which in turn transfers the force to the front bumper 70. The front bumper 70 transfers the force to the anterior section 32a of the rocker member 30, which causes the rocker member 30 to roll-up onto the foot member 10, as discussed above. Likewise, during heel-strike, the pylon 80 or other prosthesis applies a rearward force onto the housing 52 of the ankle module 50. The wall 52a of the housing hydraulically transfers said force to the posterior piston 56 when the valve 58 is closed, and the posterior piston 56 transfers the force to the rear bumper 76. The rear bumper 76 then transfers the force to the posterior section 34a of the rocker member 30, which transfers it to the posterior section 14a of the foot member 10.

For purposes of summarizing the invention and the advantages achieved over the prior art certain objects and advantages of the invention have been described hereinabove. Of course it is to be understood that not necessarily all such objects or advantages may be achieve in accordance with any particular embodiment of the invention. Thus, for example, those skilled in the art will recognize that the invention may be embodied but carried out in a manner that achieves or optimizes one advantage or a group of advantages as taught herein without necessarily achieving other objects or advantages as may be taught or suggested herein. The embodiments illustrated in FIGS. 1-10 show the length L of the foot member 10 substantially coinciding with the length of the prosthetic foot 100 so that the foot member 10 extends from the toe section 2 to the heel section 4 of the foot 100. However the foot member 10 need not extend the full length of the prosthetic foot 100. In some embodiments the foot member 10 can extend to a point rearward of the toe section 2 of the prosthetic foot 100 and/or connect to another member (not shown) that extends to the toe section 2 of the foot 100. Likewise, in some embodiment the foot member 10 can extend to a point frontward of the heel section 4 of the prosthetic foot 100 and/or connect to another member (not shown) that extends to the heel section 4 of the foot 100.

All of these aspects are intended to be within the scope of the invention herein disclosed. These and other aspects of the present invention will become readily apparent to those skilled in the art from the appended claims and from the preceding detailed description of the preferred embodiments having referenced the attached figures, the invention not being limited to any preferred embodiments disclosed.

1. A prosthetic foot comprising:
a foot member having a toe section, an intermediate section and a heel section, and defining a length between the toe section and the heel section;
a rocker member having a posterior section and an elongated anterior section and defining a length therebetween, the posterior section connected to the heel section, the anterior section configured to move relative to the foot member;
an ankle module pivotally connected to the rocker member, the ankle module having an anterior portion and a posterior portion;
a front bumper disposed between the anterior portion of the ankle module and the anterior section of the rocker member; and
a rear bumper disposed between the posterior portion of the ankle module and the posterior section of the rocker member.

2. The prosthetic foot of claim 1, wherein the front bumper is removably disposed between the anterior portion of the ankle module and the anterior section of the rocker member.

3. The prosthetic foot of claim 1, wherein at least a portion of the front bumper is configured to compress when the anterior portion of the ankle module moves toward the anterior section of the rocker member.

4. The prosthetic foot of claim 1, wherein the rear bumper is removably disposed between the posterior portion of the ankle module and the posterior section of the rocker member.

5. The prosthetic foot of claim 1, wherein at least a portion of the rear bumper is configured to compress when the posterior portion of the ankle module moves toward the posterior section of the rocker member.

6. The prosthetic foot of claim 1, wherein the rocker member has a recessed surface.

7. The prosthetic foot of claim 1, wherein the anterior section of the rocker member is tapered.

8. The prosthetic foot of claim 1, wherein the posterior section of the rocker member is tapered.

9. The prosthetic foot of claim 1, wherein the rocker member defines a roll-up surface defining a longitudinal slot between the rocker member and the foot member.

10. The prosthetic foot of claim 9, wherein the roll-up surface is inclined relative to the foot member.

11. The prosthetic foot of claim 10, wherein the roll-up surface is inclined at an angle of between about 10 degrees and about 20 degrees.

12. The prosthetic foot of claim 9, wherein the roll-up surface has a length of between about 10% and about 70% of the length of the rocker member.

13. The prosthetic foot of claim 1, wherein the length of the rocker member is greater than about 50% of the length of the foot member.

14. A prosthetic foot comprising:
a foot member having a toe section, an intermediate section and a heel section, wherein the heel section has a thickness that tapers from the intermediate section toward the heel section; and
a rocker member having an anterior section and a posterior section, the posterior section connected to the heel section, the anterior section configured to move relative to the foot member for increased flexion of the foot member, wherein the tapered thickness of the heel section of the foot member facilitates the motion of the rocker member relative to the foot member.

15. The prosthetic foot of claim 14, wherein at least a portion of the anterior section of the rocker member is a roll-up surface and defines a longitudinal slot between the rocker member and the foot member.

16. The prosthetic foot of claim 15, wherein the roll-up surface is inclined relative to the foot member.

17. The prosthetic foot of claim 16, wherein the roll-up surface is inclined at an angle of between about 10 degrees and about 20 degrees.

18. The prosthetic foot of claim 16, wherein the roll-up surface has a length of between about 10% and about 70% of the length of the rocker member.

19. The prosthetic foot of claim 14, wherein the anterior section of the rocker member is tapered to facilitate insertion into and removal from a cosmesis.

20. The prosthetic foot of claim 14, wherein the rocker member is removably connected to the heel section.

21. The prosthetic foot of claim 14, further comprising an ankle module pivotally connected to the rocker member, the ankle module having a housing configured to slidingly receive an anterior piston and a posterior piston, the ankle module also having a pyramid configured to receive a pylon thereon and an actuator configured to receive a force, wherein selective actuation of the actuator allows the housing to be selectively moved relative to the pistons.

22. The prosthetic foot of claim 21, further comprising a front bumper removably disposed between the anterior piston of the ankle module and the anterior section of the rocker member, wherein at least a portion of the front bumper is configured to compress.

23. The prosthetic foot of claim 21, further comprising a rear bumper removably disposed between the posterior piston of the ankle module and the posterior section of the rocker member, wherein at least a portion of the rear bumper is configured to compress.

24. The prosthetic foot of claim 21, wherein the pyramid is configured to be selectively positioned at at least one angle relative to a support surface upon application of the force to said actuator, said positioning at at least one angle varying a height of the heel section relative to the support surface when the pyramid is oriented generally orthogonal to the support surface.

25. A prosthetic foot comprising:
a foot member;
a rocker member connected to the foot member, the rocker member including at least one surface capable of moving relative to the foot member when the prosthetic foot is in use;
an ankle module pivotally connected to the rocker member, and
at least one bumper disposed between the ankle module and the rocker member.

26. The prosthetic foot of claim 25, wherein the at least one bumper includes a front bumper and a rear bumper.

27. The prosthetic foot of claim 26, wherein the front bumper is positioned anterior to a pivot point between the ankle module and the rocker member.

28. The prosthetic foot of claim 25, wherein the foot member extends from a heel section to a toe section.

29. The prosthetic foot of claim 25, wherein the rocker member is connected solely at a posterior section thereof to a posterior section of the foot member.

30. The prosthetic foot of claim 25, wherein at least a portion of an anterior section of the rocker member is a roll-up surface and defines a longitudinal slot between the rocker member and the foot member.

31. The prosthetic foot of claim 30, wherein the roll-up surface is inclined relative to the foot member.

32. The prosthetic foot of claim 31, wherein the roll-up surface is inclined at an angle of between about 10 degrees and about 20 degrees.

33. The prosthetic foot of claim 30, wherein the roll-up surface has a length of between about 10% and about 70% of the length of the rocker member.

34. The prosthetic foot of claim 25, wherein the rocker member defines a length between the front end and a rear end thereof, and wherein said length of the rocker member is greater than about 50% of the length of the foot member.

35. A prosthetic foot comprising:
a foot member;
a rocker member having a front end extending forward of a midline of the foot member, the rocker member including at least one surface capable of moving relative to the foot member when the prosthetic foot is in use.

36. The prosthetic foot of claim 35, further comprising an ankle module pivotally connected to the rocker member.

37. The prosthetic foot of claim 36, further comprising at least one bumper disposed between the ankle module and the rocker member.

38. The prosthetic foot of claim 37, wherein the at least one bumper includes a front bumper and a rear bumper.

39. The prosthetic foot of claim 38, wherein the front bumper is positioned anterior to a pivot point between the ankle module and the rocker member.

40. The prosthetic foot of claim 35, wherein the rocker member defines a length between the front end and a rear end thereof, and wherein said length of the rocker member is greater than about 50% of the length of the foot member.

41. A prosthetic foot comprising:
a foot member having a toe section and a heel section;
a rocker member connected to the foot member, the rocker member including at least one surface capable of moving relative to the foot member when the prosthetic foot is in use; and
an ankle module operatively connected to the foot member and selectively adjustable to change the height of the heel section relative to a support surface that operatively contacts the foot member.

42. The prosthetic foot of claim 41, wherein the ankle module has an actuator configured to selectively lock the module in a desired position, and configured to selectively allow the ankle module to be repositioned relative to the foot member to change the height of the heel section.
